(19) United States
(12) Patent Application Publication
(10) Pub. No.: US 2005/0089859 A1
(43) Pub. Date: Apr. 28, 2005

(54) Method for the isolation of nucleic acids

(76) Inventors: Thorsten Singer, Solingen (DE); Martin Weber, Leichlingen (DE)

Correspondence Address: Leon R Yankwich;Yankwich & Associates, 201 Broadway, Cambridge, MA, 02139, US

(21) Appl. No.: 10/493,925

(22) PCT Filed: Nov. 6, 2002
(86) PCT No.: PCT/EP02/12384

(30) Foreign Application Priority Data
Nov. 6, 2001 (DE) 101 53 957.6

(51) Int. Cl. C12Q 1/68; C07H 21/04

(52) U.S. Cl. 435/6; 536/25.4

(57) ABSTRACT

The present invention relates to a simplified fast process for isolating nucleic acids, particularly plasmid DNA from E. coli.

The present invention relates to a simplified fast process for isolating nucleic acids, particularly plasmid DNA from E. coli.

Methods of isolating nucleic acids from complex starting materials are known per se from the prior art and comprise in the first step lysing the biological material using a detergent. This first step is carried out in the presence of enzymes which are necessary for breaking down protein.

In a second step there is generally extraction of the nucleic acids with suitable organic solvents such as, for example, phenol and/or chloroform. This step is generally followed by precipitation of the nucleic acids with ethanol and dialysis of the nucleic acids. Thus, a process is known from the prior art in which plasmid DNA is subjected to various extraction and detergent treatments in order to lyse the cells from which the plasmid nucleic acid is then obtained and in order to eliminate cell constituents and other nucleic acid material. In a final step the pure plasmid DNA is bound to ground glass in the absence of a chaotropic substance [M. A. Marko et al., Analytical Biochemistry 121, 382-387,1982].

The processes known from the prior art, such as the isolation of double-stranded DNA, for example, are very laborious and time consuming, however. The relatively large number of steps required to isolate nucleic acids from starting materials of this kind increase the risk of contamination when a number of samples are being dealt with simultaneously. This situation presents an almost incalculable risk, particularly when dealing with clinical samples. If the nucleic acid is to be isolated for subsequent examination for the presence of nucleic acid, e.g. of a pathogen (e.g. a virus or a bacterium) by the nucleic acid amplification process, e.g. by polymerase chain reaction [PCR, Saiki et al., Science 230, 1985 1350] this risk of contamination with foreign nucleic acid is totally unacceptable.

Moreover, alternative methods are known from the prior art but these are also associated with the risk of sample contamination.

Thus, a process for isolating total RNA from tissues and cell cultures is known from the prior art [Analytical Biochemistry 162, 1987, 156]. According to this process the RNA is removed from the biological starting material in a single guanidinium thiocyanate/phenol/chloroform mixture. After phase separation the RNA can be obtained in a usable state within 4 hours by further processing of the aqueous phase.

The prior art also describes a process for isolating DNA from tissues and cell lines in which the cells are dispersed in a buffer containing guanidinium-HCl and are precipitated in ethanol [Analytical Biochemistry 162, 1987, 463]. It is known of this process that it is prone to contamination, but a usable NA product can be isolated within a few hours after working up the separated DNA.

Particularly with respect to processes for isolating plasmid DNA by means of a plasmid matrix it can be established that such processes are generally based on alkaline lysis (resuspension, alkaline lysis, neutralisation), the neutralisation buffer containing chaotropic salts for creating the conditions for binding the DNA to the silica matrix. The disadvantage of these methods known from the prior art is that a voluminous precipitate is obtained, with the result that the lysate has to be clarified in another highly time consuming step (e.g. centrifugation or filtration) before it can be brought into contact with the matrix.

The lysate thus clarified is then transferred into a column and processed by the application of a vacuum or by centrifugation through the silica membrane.

In order to remove impurities the membrane is washed with a buffer which contains alcohol and within the course of a subsequent centrifugation/vacuum treatment any alcohol residues are eliminated. Finally, the nucleic acid (DNA) is eluted with a so-called low salt buffer (e.g. 10 mM Tris-HCl, pH 8.5). The underlying principle of this method is disclosed, inter alia, in European Patent Application No. 90 200 678.2 and by R. Boom et al. [R. Boom et al. J. Clin. Microbiol, 28 (3) (1990) 495].

The present invention therefore sets out to overcome the disadvantages of the processes known from the prior art and in particular to provide a process which does not require time-consuming clarification of the lysate.

Another objective of the present invention is to provide a process which largely manages without any substances which are regarded as “irritant” or even damaging to health.

According to the invention these objectives are achieved by using a neutralising buffer based on a salt of a carboxylic acid for the neutralising step which follows the known alkaline lysis with a buffer known for this purpose from the prior art (e.g. 200 mM NaOH, 1% SDS).

Surprising, it has been found that by using the process according to the invention the yield is also increased compared with the corresponding processes known from the prior art.

Examples of carboxylic acids for the purposes of the invention include, first of all, saturated aliphatic monocarboxylic acids, preferably C₁-C₆-alkylcarboxylic acids, including acetic acid, propionic acid, n-butyric acid, n-valeric, isovaleric acid, ethyl-methyl-acetic acid (2-methylbutyric acid), 2,2-dimethylpropionic acid (pivalic acid), n-hexanoic acids. Salts of formic acid and/or acetic acid are most preferably used.

Examples of unsaturated alkenylcarboxylic acids for the purposes of the invention include acrylic acid (propionic acid), methacrylic acid, crotonic acid, iso-crotonic acid and vinylacetic acid.

In addition, saturated aliphatic C₂-C₆-dicarboxylic acids may be used, such as, for example, oxalic acid, malonic acid, succinic acid, glutaric acid or adipic acid.

Suitable acids for the purposes of the invention include aliphatic hydroxy-di- and tri-carboxylic acids, of which (2R,3R)-(+)-tartaric acid, (2R,3R)-(−)-tartaric acid or meso-tartaric acid are preferred.

According to the invention, salts of the alkali metals, ammonium salts or salts or quaternary amines are used, in particular.

It is particularly preferable to use lithium or sodium or ammonium (NH₄⁺) salts.

In addition, mixtures of the salts described may be used.

By using aqueous solutions of these salts clouding of the lysate is avoided according to the invention, thus dispensing with the need for a subsequent clarification step.

For example, the process according to the invention for isolating DNA from E. coli is as follows:

The pellet obtained from the bacterial culture is first resuspended in a suitable buffer. Buffers of this kind are known from the prior art and are commercially available. A suitable buffer consists of an aqueous solution containing 50 mM Tris and 10 mM EDTA (pH 8.8). The buffer specified is conventionally used for resuspending bacteria, but it is also possible to use any other salts which buffer in the region of a physiological pH.

The buffers which may be used for the subsequent lysing are also known from the prior art and commercially obtainable. A suitable buffer consists of a 200 mM sodium hydroxide solution containing 1% SDS—here too it is possible to use standard commercial buffers which optionally contain other detergents such as Triton, for example.

After lysing over a sufficiently long period, an aqueous solution of an acetate salt is added according to the invention, e.g. a 3 M aqueous ammonium acetate solution (pH 5.5). It is also possible to use derivatised acetates, such as halogen-substituted acetates, for example.

After the addition of an alcohol, preferably a straight-chain or branched C₁ to C₃ alcohol, more preferably iso-propanol, the mixture obtained is brought into contact with a silica matrix, while the DNA may be quantitatively bound.

The process according to the invention may additionally be carried out with other compounds which contain alcoholic hydroxy groups, such as, for example, polyethylene glycols. Preferably polyethylene glycols with a molecular weight within the range from 2,000 to 10,000, more preferably with a molecular weight from 4,000 to 8,000 are used for this.

The separation of the proteins, when using a silica membrane as matrix, is achieved by the fact that the protein fraction which is in solution does not bind to the silica matrix, whereas precipitated proteins are irreversibly denatured and proteins are retained by the filtration effect when passed through the membrane.

After the crude lysate has been passed through the silica matrix, the membrane is washed with a washing buffer, for example.

Suitable washing buffers are also known from the prior art and are commercially available. The only requirement of suitable buffers is that the buffer must ensure that the nucleic acid is not detached from the matrix. Generally speaking, a high alcohol content and optionally a slightly alkaline pH are sufficient to prevent autoproteolysis of the DNA. Buffers which contain chaotropic compounds, such as PB, for example (QIAGEN GmbH, Hilden, Federal Republic of Germany), are also suitable provided that they meet the conditions mentioned above.

After the removal of the buffer residues, e.g. by centrifugation—the nucleic acid is finally eluted using a suitable elution buffer. These elution buffers are also fairly well known from the prior art and are commercially available. It is preferable to use buffers with a low salt concentration or water.

### EXPLANATION OF THE FIGURES

FIG. 1 shows the agarose gel from Example 2 on which the densitometric measurements are based.

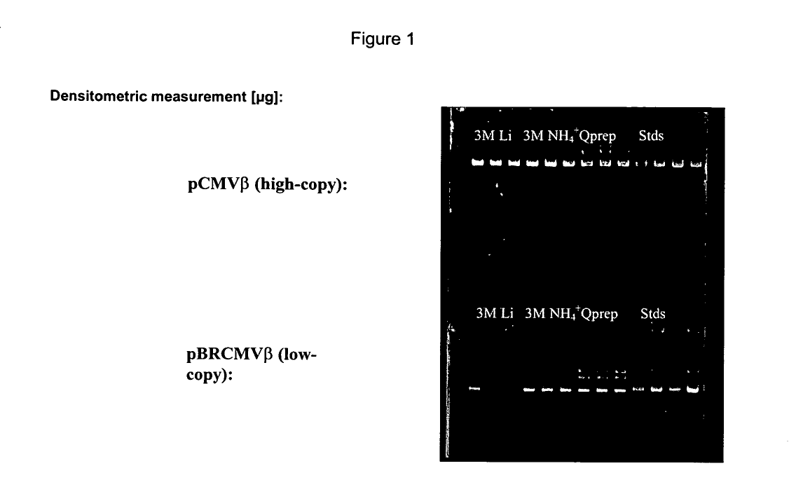

FIG. 2 shows the agarose gel from Example 3 on which the densitometric measurements are based: equal volumes of preparation 1 have been applied in each case.

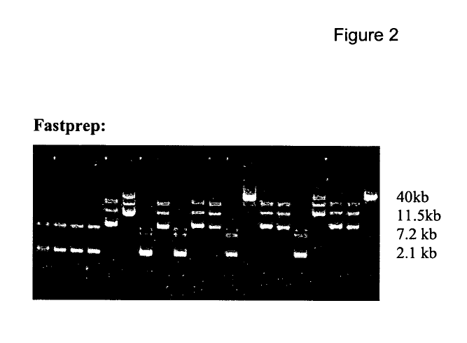

FIG. 3 shows the agarose gel from Example 4 on which the densitometric measurements are based.

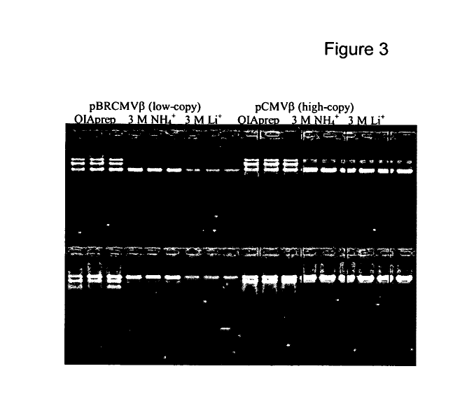

FIG. 4 shows the agarose gels from Example 8 on which the densitometric yield measurements are based.

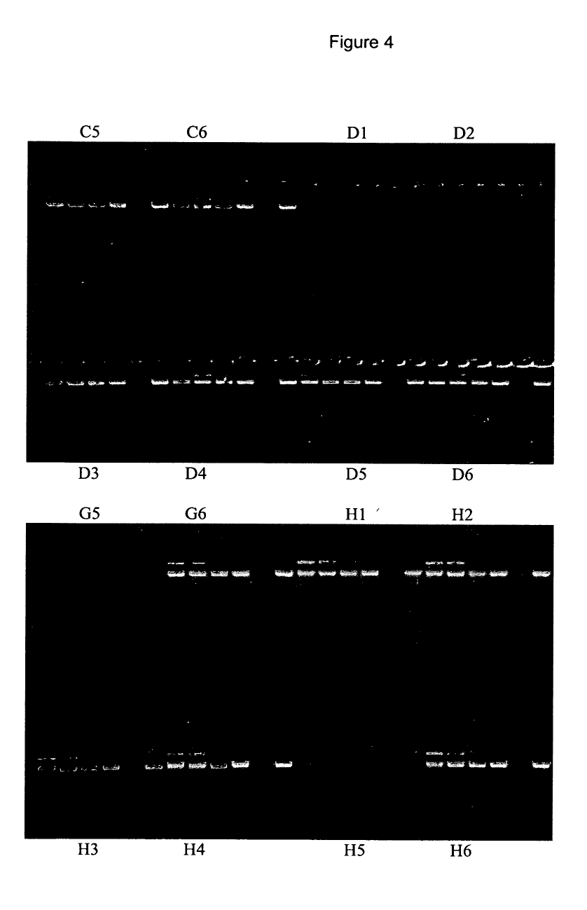

Sequence of the samples (from left to right):
Process according to the invention—96-1 BR 3000
Process according to the invention—96-2 BR 3000
Prior art (QIAprep Turbo 96) BR 3000
Process according to the invention—96-1 manual—dummy
Process according to the invention—96-2 manual.

The names in the margin indicate the position of the samples applied in the 96 well block in accordance with the SBS standard.

The gels exhibit equivalent results for all the preparation methods. The failure of wells G5 and H5 is due to the heterogeneity of the gene bank used and are independent of the preparation method used.

The Examples which follow are intended to illustrate the invention.

Preliminary Remarks:

The Examples provided were carried out according to the following procedure. Any deviations from this are specially mentioned.

Method of Isolating Plasmid DNA from E. coli on a Small Scale:

For 1.5 ml of E. coli Culture
(1) Resuspend the bacterial pellet in 150 μl of resuspension buffer
(2) Add 150 μl of lysis buffer and mix carefully. Lyse for about 3 minutes
(3) Add 150 μl of neutralising buffer and mix thoroughly (do not vortex!)
(4) Add 300 μl of isopropanol and mix thoroughly (do not vortex!)
(5) Pass the crude lysate through a silica membrane column and centrifuge for 1 minute at 14000 rpm
(6) Wash by adding 750 μl of 80% by volume aqueous ethanol and 10 mm Tris and centrifuge for 1 minute at 14000 rpm
(7) In order to eliminate traces of buffer centrifuge for 1 minute at 14000 rpm
(8) Elute with 200 μl of 10 mm Tris solution (pH 8.5). Pipette onto the membrane, leave to stand for 1 minute and centrifuge thoroughly (1 minute at 14000 rpm).

By omitting the lysate clarification step the preparation time is reduced by 10 minutes to half the time.

In the examples provided, reference preparations were carried out with QIAprep as a representative example of a standard commercial silica method with a chaotropic binding buffer. The reference is characterised by “QIAprep” in the examples provided (in the QIAprep method the DNA from a previously clarified lysate is bound to a silica membrane in the presence of chaotropic salts which are present in a high concentration and after a purification step eluted from the membrane. Suitable kits are obtainable from Messrs. QIAGEN GmbH, Hilden, Federal Republic of Germany).

### EXAMPLE 1

### Comparison of 3 M NH₄OAc, pH 5.5, with 3 M LiOAc (Ac=COCH₃), pH 5.5 as Neutralising Buffer; Effect of the Cation

As a reference, a plasmid isolation was carried out in parallel with QIAprep.

DH5a/pCMVβ (high-copy):

| | | |
|---|---|---|
| 3 M Li | 3 M NH₄⁺ | QIAprep |

OD₂₆₀ [μg]:

| | | | |
|---|---|---|---|
| 1 | 11.1 | 12.4 | 18.1 |
| 2 | 10.8 | 10.5 | 16.8 |
| 3 | 11.8 | 11.2 | 17.2 |
| X | 11.2 | 11.4 | 17.4 |

Densitometric measurement [μg]:

| | | | |
|---|---|---|---|
| 1 | 11.6 | 14.0 | 14.4 |
| 2 | 12.0 | 11.7 | 15.7 |
| 3 | 11.6 | 10.2 | 15.6 |
| X | 11.7 | 12.0 | 15.2 |

Neutralisation buffer used (Step 3 in the procedure described above):
3 M Li 3 M Li-acetate, pH 5.5
3 M NH₄⁺ 3 M NH₄⁺-acetate, pH 5.5

The results show that in high-copy plasmids with both neutralising buffers a high degree of conformity is achieved between the two methods of quantification. In other words primarily only plasmid DNA is isolated.

DH5a/pBRCMVβ (low-copy):
OD₂₆₀ [μg]:

| | 3 M Li | 3 M NH₄⁺ | QIAprep |
|---|---|---|---|
| 1 | 5.1 | 3.6 | 3.8 |
| 2 | 11.5 | 3.4 | 3.5 |
| 3 | 17.5 | 4.0 | 3.1 |
| X | 11.4 | 3.7 | 3.5 |
| 1 | 0.7 | 1.8 | 2.7 |
| 2 | 0.9 | 2.0 | 3.0 |
| 3 | 0.4 | 2.1 | 2.7 |
| X | 0.7 | 2.0 | 2.8 |

In contrast to the high-copy plasmids the low-copy preparations show distinct differences between neutralisation with Li-acetate and NH₄-acetate. Good conformity between the two methods of measurement is only achieved on neutralisation with ammonium acetate. This state of affairs proves that the system is consequently more robust and is therefore particularly suitable for general use.

### EXAMPLE 2

### Comparison of 3 M NH₄OAc, pH 5.5 with Other Ammonium Salt Solutions as Neutralisation Buffer; Influence of the Anion

| Anion | formate | | HPO₄²⁻ | | SO4²⁻ | | tartrate | H₂PO₄⁻ |
|---|---|---|---|---|---|---|---|---|
| conc. | 1 M | 3 M | 1 M | 3 M | 1 M | 3 M | 1 M | 1 M |

Yields [μg] according to OD₂₆₀:

| | | | | | | | | |
|---|---|---|---|---|---|---|---|---|
| 1 | 3.3 | 4.7 | 2.3 | 3.3 | 2.1 | 3.0 | 2.8 | 2.5 |
| 2 | 2.4 | 4.0 | 2.4 | 3.6 | 2.5 | 2.3 | 2.1 | 2.3 |
| X | 2.9 | 4.4 | 2.4 | 3.5 | 2.3 | 2.7 | 2.5 | 2.4 |

Yields [μg] according to densitometry:

| | | | | | | | | |
|---|---|---|---|---|---|---|---|---|
| 1 | 1.3 | 4.8 | 1.8 | 1.0 | 1.8 | 0 | 2.8 | 0.6 |
| 2 | 0.8 | 4.5 | 1.3 | 1.4 | 2.1 | 0 | 1.7 | 0.3 |
| X | 1.1 | 4.7 | 1.6 | 1.2 | 2.0 | 0 | 2.3 | 0.5 |

X = average

The reference yields with 3 M NH₄OAc, pH 5.5 were taken as 0% false quantification and were 5.2 μg on average (two separately prepared buffer charges, each measured twice).

The results shown above demonstrate the influence of the anion both in terms of the total yields and in reducing the photometric overquantification.

### EXAMPLE 3

### Comparison of the Process According to the Invention (DirectPrep) for Isolating Various Constructs from Various Strains of E. coli

For this, four typical laboratory strains were selected and plasmids of different copy numbers and different sizes were isolated from them.

| | size | | | | | | |
|---|---|---|---|---|---|---|---|
| Plasmid | [kb] | strain | OD₆₀₀ | 1 | 2 | 3 | X |

Process according to the invention:

| | | | | | | | |
|---|---|---|---|---|---|---|---|
| Cosmid 9 | 40 | DH5a | 2.8 | 10 | 8.3 | 7.4 | 8.6 |
| (low-copy) | | HB101 | 3.2 | 8.1 | 9.1 | 9.2 | 8.8 |
| pBRCMVβ | 6.8 | DH5a | 3.4 | 7 | 4.8 | 4.9 | 5.6 |
| (low-copy) | | HB101 | 3.4 | 5.9 | 4.1 | 5.6 | 5.2 |
| | | TOP10F′ | 2.5 | 2.6 | 2.7 | 2.5 | 2.6 |
| | | XL1blue | 3.5 | 5.5 | 5.4 | 5.7 | 5.5 |
| pCMVβ | 7.2 | DH5a | 2.3 | 12.1 | 10.2 | 11.8 | 11.4 |
| (high-copy) | | HB101 | 2.6 | 8.2 | 9.9 | 8.3 | 8.8 |
| | | TOP10F′ | 2.7 | 9.5 | 11.3 | 10.8 | 10.5 |
| | | XL1blue | 2.1 | 10.8 | 11.9 | 11.8 | 11.5 |
| pTS64 | 11.5 | DH5a | 2.1 | 9.9 | 9.3 | 9.9 | 9.7 |
| (high-copy) | | XL1blue | 2.4 | 5.1 | 14.8 | 20.6 | 13.5 |
| pUC21 | 3.2 | DH5a | 2.8 | 4.6 | 4.5 | 4.7 | 4.6 |
| (high-copy) | | HB101 | 3.2 | 4.8 | 5.5 | 4.1 | 4.8 |
| | | TOP10F′ | 2.1 | 3.4 | 3.8 | 3.7 | 3.6 |
| | | XL1blue | 1.9 | 4.1 | 4 | 4.4 | 4.2 |

Prior art process (QIAprep):

| | | | | | | | |
|---|---|---|---|---|---|---|---|
| Cosmid 9 | 40 | DH5a | 2.8 | 10.9 | 10.8 | 9.8 | 10.5 |
| (low-copy) | | HB101 | 3.2 | 11.3 | 5.2 | 11.5 | 9.3 |
| pBRCMVβ | 6.8 | DH5a | 3.4 | 4.1 | 4.2 | 4.7 | 4.3 |
| (low-copy) | | HB101 | 3.4 | 6 | 9.2 | 8.6 | 7.9 |
| | | TOP10F′ | 2.5 | 3.8 | 3.7 | 3.8 | 3.8 |
| | | XL1blue | 3.5 | 5.7 | 5.7 | 4.8 | 5.4 |
| pCMVβ | 7.2 | DH5a | 2.3 | 14.7 | 13.6 | 14.2 | 14.2 |
| (high-copy) | | HB101 | 2.6 | 10.3 | 10.6 | 9.9 | 10.3 |
| | | TOP10F′ | 2.7 | 18.9 | 18.7 | 18.1 | 18.6 |
| | | XL1blue | 2.1 | 15.9 | 14.5 | 13.9 | 14.8 |
| pTS64 | 11.5 | DH5a | 2.1 | 14.1 | 13.4 | 14.1 | 13.9 |
| (high-copy) | | XL1blue | 2.4 | 24.6 | 28.1 | 23.5 | 25.4 |
| pUC21 | 3.2 | DH5a | 2.8 | 5.6 | 5.6 | 5.6 | 5.6 |
| (high-copy) | | HB101 | 3.2 | 4.1 | 4.8 | 4.7 | 4.5 |
| | | TOP10F′ | 2.1 | 5.1 | 5.2 | 5 | 5.1 |
| | | XL1blue | 1.9 | 6.4 | 5.8 | 5.7 | 6.0 |

In order to determine overquantification all the QIAprep values were taken to be 100%, i.e. total agreement between the OD₂₆₀ and densitometric measurement was assumed. The following relative values were thus obtained for the fast preparation:

| Plasmid | size [kb] | strain | OD₆₀₀ | densitometry |
|---|---|---|---|---|
| Cosmid 9 | 40 | DH5a | 82 | 99 |
| (low-copy) | | HB101 | 94 | 93 |
| pBRCMVβ | 6.8 | DH5a | 128 | 86 |
| (low-copy) | | HB101 | 66 | 120 |
| | | TOP10F′ | 69 | 82 |
| | | XL1blue | 102 | 90 |
| pCMVβ | 7.2 | DH5a | 80 | 119 |
| (high-copy) | | HB101 | 86 | 93 |
| | | TOP10F′ | 57 | 77 |
| | | XL1blue | 78 | 75 |
| pTS64 | 11.5 | DH5a | 70 | 100 |
| (high-copy) | | XL1blue | 53 | 64 |
| pUC21 | 3.2 | DH5a | 82 | 111 |
| (high-copy) | | HB101 | 106 | 130 |
| | | TOP10F′ | 71 | 101 |
| | | XL1blue | 70 | 94 |
| Averages: | | | 80 | 97 |

On average a yield of 80% of the yields of the QIAprep preparations was obtained according to OD₂₆₀ and a yield of almost 100% was obtained according to densitometric evaluation.

The Example shows that the new process described is generally applicable and gives virtually identical results compared with established methods of the prior art.

The agarose gels also demonstrate a further advantage of the process according to the invention: the DNA showed significantly less shearing of the plasmids than in the previous processes and almost 100% of it is present in supercoiled form.

### EXAMPLE 4

### Stability and Quality of the Isolated Plasmid DNA

A high-copy plasmid and a low-copy plasmid were isolated in a triple measurement and incubated for 20 h at 37° C. in the presence of a DNase reaction buffer (the composition of such buffers is known from the prior art, e.g. from standard molecular biology textbooks).

No difference could be found between incubated and non-incubated plasmid DNA, proving that the preparation contained no DNAses.

This Example clearly shows that DNA isolated by the process according to the invention is free from contaminating DNAses and the stability on storage is equivalent to the established silica processes with clarification of the lysate and chaotropic binding chemistry. (Reference for an established method of preparation: QIAprep, Messrs. QIAGEN). In contrast to the established method the DNA isolated by the new process has a much larger proportion of intact supercoiled form.

Test sequences yielded identical reading frames for plasmid DNA isolated by the new process and the QIAprep method.

### EXAMPLE 5

### Dependency on the Quantity of Isopropanol Used for Binding

1.5 ml aliquots of DH5α/pBRCMVβ (low-copy) were worked up and increasing amounts of isopropanol were used to bind the plasmid DNA.

| | | | | | | | |
|---|---|---|---|---|---|---|---|
| Vol % Isoprop. | 28.6 | 33.3 | 37.5 | 41.2 | 44.4 | 47.4 | 50 |

Yields [μg] according to OD₂₆₀:

| | | | | | | | |
|---|---|---|---|---|---|---|---|
| 1 | 2.2 | 2.6 | 2.8 | 2.9 | 4.1 | 5.5 | 14.1 |
| 2 | 3.8 | 2.2 | 2.7 | 3.1 | 6.0 | 9.3 | 18.2 |
| 3 | 2.5 | 2.6 | 2.4 | 3.5 | 5.4 | 7.0 | 13.7 |
| Average | 2.8 | 2.5 | 2.6 | 3.2 | 5.2 | 7.3 | 15.3 |

Yields [μg] according to densitometry:

| | | | | | | | |
|---|---|---|---|---|---|---|---|
| 1 | 1.1 | 2.6 | 2.8 | 2.9 | 2.8 | 2.1 | 4.3 |
| 2 | 2.7 | 1.8 | 3.2 | 2.9 | 3.9 | 3.8 | 4.0 |
| 3 | 2.4 | 2.9 | 2.4 | 2.6 | 3.1 | 3.3 | 3.6 |
| Average | 2.1 | 2.4 | 2.8 | 2.8 | 3.3 | 3.1 | 4.0 |

If the values obtained from photometric and densitometric analysis are compared, it will be seen that there is a gradual increase in the yield as the proportion of isopropanol increases. At the same time it is noticeable that this increase is much greater with photometric measurement than in densitometry. As the densitometric measurement only includes the plasmid DNA present, whereas photometric analysis picks up all the nucleic acids, the increasing discrepancy between OD measurement and densitometry with larger amounts of isopropanol shows a higher level of contamination. The standard 41.2% by volume used in the process are evidence of a very high similarity between the two methods of measurement with a higher total yield compared with mixtures containing smaller amounts of alcohol. This shows that the plasmid DNA isolated in this way contains a smaller amount of other contaminating nucleic acids.

### EXAMPLE 6

### Effect of the Alcohol Used

1.5 ml aliquots of DH5α/pCMVβ (Messrs. Clontech) (high-copy) were worked up and C₁-C₄-alcohols were tested for their suitability for binding plasmid DNA. All amounts given in μg.

| Alcohol | Isoprop | EtOH | MeOH | 1-ButOH | 1-ButOH | 2-ButOH | 2-ButOH | QIAprep |
|---|---|---|---|---|---|---|---|---|
| Vol % | 37.5 | 50 | 50 | 50 | 33.3 | 50 | 33.3 | — |

Yield OD₂₆₀

| | | | | | | | | |
|---|---|---|---|---|---|---|---|---|
| 1 | 10.5 | 10.0 | 7.7 | 2.1 | 5.9 | 2.4 | 5.4 | 11.3 |
| 2 | 9.2 | 11.0 | 6.7 | 4.5 | 3.6 | 3.4 | 3.8 | 11.0 |
| X | 9.9 | 10.5 | 7.2 | 3.3 | 4.8 | 2.9 | 4.6 | 11.1 |

Yield densitometry

| | | | | | | | | |
|---|---|---|---|---|---|---|---|---|
| 1 | 11.7 | 11.4 | 3.6 | 0.4 | 1.0 | 0.3 | 2.0 | 9.0 |
| 2 | 10.0 | 11.8 | 4.5 | 0.7 | 0.6 | 1.2 | 1.6 | 9.1 |
| X | 10.8 | 11.6 | 4.0 | 0.6 | 0.8 | 0.8 | 1.8 | 9.0 |

OD-overquantification [%]

| | | | | | | | | |
|---|---|---|---|---|---|---|---|---|
| 1 | 90 | 88 | 214 | 488 | 583 | 713 | 270 | 126 |
| 2 | 92 | 94 | 150 | 652 | 587 | 287 | 246 | 121 |
| X | 91 | 91 | 178 | 588 | 584 | 381 | 259 | 124 |

Isoprop Isopropanol
EtOH Ethanol
MeOH Methanol
1-ButOH Butan-1-ol
2-ButOH Butan-2-ol

The results clearly show that isopropanol and ethanol and also methanol are suitable for selectively binding plasmid DNA to the silica matrix in order to carry out the process according to the invention.

### EXAMPLE 7

### Use of polyethyleneglycols for Adjusting the Binding Conditions

1.5 ml aliquots of DH5α/pCMVβ (Messrs Clontech) (high-copy) were worked up and polyethyleneglycols of different molecular weights were tested for their suitability for binding plasmid DNA. 300 μl of a 40% (w/v) solution were used, in accordance with the procedure described hereinbefore. All amounts are given in μg.

| | | | |
|---|---|---|---|
| PEG | 4000 | 6000 | 8000 |

Yield OD₂₆₀

| | | | |
|---|---|---|---|
| 1 | 4.6 | 3.5 | 5.7 |
| 2 | 4.6 | 5.1 | 5.3 |
| X | 4.6 | 4.3 | 5.5 |

Yield Densitometer

| | | | |
|---|---|---|---|
| 1 | 4.4 | 2.3 | 2.8 |
| 2 | 4.0 | 4.1 | 5.0 |
| X | 4.2 | 3.2 | 3.9 |

X = average
PEG = Polyethylene Glycol
4000, 6000, 8000 = average molecular weight of the polyethyleneglycols

The experimental findings show that even polyethyleneglycols of different molecular weight are also very suitable for adjusting the binding conditions within the scope of the process according to the invention. The yields achieved and the degree of conformity between photometric and densitometric quantification are comparable to the results obtained with isopropanol, for example.

### EXAMPLE 8

### Use of the Process Described in the High Throughput Range

1.25 ml aliquots of DH10B/pUC19 gene bank (high-copy) were cultured in a 96 well block and worked up according to the procedure described under “Preliminary remarks”. In order to bind the DNA a 96 well plate with a suitable membrane combination was used. The process was carried out manually and using a BIOROBOT (Messrs. QIAGEN).

The reference used, the QIAprep Turbo System (QIAGEN GmbH, Hilden, FGederal Republic of Germany), is a method which has long been on the market.

All the results are given in pg. Yields are averaged over all 96 samples in each case.

Yield Densitometer

| | | |
|---|---|---|
| manual | process according to the invention 96 | 4.3 |
| | prior art process (QIAprep Turbo 96) | 6.0 |
| BR3000 | process according to the invention 96 | 3.0 |
| | prior art process (QIAprep Turbo 96) | 2.8 |

BR3000 . . . BIORobot 3000 (QIAGEN GmbH, Hilden, Federal Republic of Germany).

To test the quality of the DNA isolated, 24 of the 96 samples were sequenced and subjected to Phred20 analysis, which indicates the reading lengths obtained and hence the quality.

| | |
|---|---|
| Prior art (QIAprep Turbo): | 524 base pairs |
| Process according to the invention: | 505 base pairs |

Allowing for the usual fluctuations which occur during isolation from biological systems, the process according to the invention has proved just as good as the processes known from the prior art in terms of yield and quality. However, the omission of the lysate clarification step makes it easy to simultaneously prepare 96 clones per plate, in contrast to the prior art. Therefore, the process according to the invention is particularly suitable for the high throughput range, both manually and fully automatically.

The present invention also relates to a formulation or a kit for carrying out one of the processes as claimed, particularly a kit containing the alkali metal salt—especially the lithium or sodium—or ammonium salt of an aliphatic carboxylic acid—preferably formic or acetic acid—optionally in aqueous solution; an aliphatic alcohol, particularly iso-propanol and/or polyethyleneglycol(s) with a molecular weight in the range from 2,000 bis 10,000; optionally a washing buffer and an eluting buffer.

1. A process for isolating nucleic acids, characterised in that a biological sample containing the nucleic acid is subjected to alkaline lysis and the resulting reaction mixture is neutralised with a salt of a carboxylic acid and the nucleic acid is then brought into contact with a silica matrix in the presence of an alcohol, the nucleic acid bound to the matrix is isolated and optionally washed with a washing buffer and the bound nucleic acid is eluted from the matrix by addition of an elution buffer to the matrix.

2. The process according to claim 1, characterised in that the salt is a salt of a saturated aliphatic monocarboxylic acid is used.

3. The process according to claim 2, characterised in that the salt is a salt of a C₁-C₆-alkyl-carboxylic acid is used.

4. The process according to claim 3, characterised in that the salt is selected from the group consisting of a salt of acetic acid, propionic acid, n-butyric acid, n-valeric acid, isovaleric acid, ethyl-methyl-acetic acid (2-methyl-butyric acid), 2,2-dimethylpropionic acid (pivalic acid), n-hexanoic acid, and combinations thereof.

5. The process according to claim 1, characterised in that the salt of a carboxylic acid is an unsaturated alkenyl-carboxylic acid is used.

6. The process according to claim 5, characterised in that the salt is selected from the group consisting of a salt of acrylic acid (propenoic acid), methacrylic acid, crotonic acid, iso-crotonic acid, vinylacetic acid, and combinations thereof ised.

7. The process according to claim 1, characterised in that the salt is a salt of a saturated aliphatic C₂-C₆-dicarboxylic acid.

8. The process according to claim 7, characterised in that the salt of said saturated aliphatic C₂-C₆ dicarboxylic acid is selected from salts of the group consisting of oxalic acid, malonic acid, succinic acid, glutaric acid and adipic acid.

9. The process according to claim 1, characterised in that the salt is the salt of an aliphatic hydroxy-di- and -tricarboxylic acid.

10. The process according to claim 9, characterised in that said salt is selected from the group consisting of the salt of an aliphatic hydroxy-di-carboxylic acid (2R,3R)-(+)-tartaric acid, (2S,3S)-(−)-tartaric acid, or meso-tartaric acid.

11. The process according to claim 1, characterised in that the salt of a carboxylic acid further includes an alkali metal.

12. The process according to claim 1, characterised in that the salt of a carboxylic acid further includes lithium acetate or sodium acetate.

13. The process according to claim 1, characterised in that the salt of a carboxylic acid further includes ammonium acetate.

14. The process according to claim 1, characterised in that the carboxylic acid salt is optionally used together with other excipients in the form of an aqueous solution.

15. The process according to claim 1, characterised in that the final concentration of the carboxylic acid salt is from 0.1 to 5M.

16. The process according to claim 15, characterised in that the final concentration of the carboxylic acid salt is from 0.3 to 2M.

17. The process according to claim 15, characterised in that the final concentration of the carboxylic acid salt in the reaction mixture is about 0.3 M.

18. The process according to claim 1, characterised in that the reaction mixture resulting from the neutralisation is brought into contact with a silica matrix in the presence of a branched or unbranched C₁-C₃-alcohol.

19. The process according to claim 18, characterised in that the alcohol is ethanol.

20. The process according to claim 18, characterised in that the alcohol is iso-propanol (propan-2-ol).

21. The process according to claim 1, characterised in that the alcohol is a polyethyleneglycol.

22. The process according to claim 21, characterised in that the average molecular weight of the polyethyleneglycol from 1,000 to 12,000.

23. The process according to claim 22, characterised in that the average molecular weight of the polyethyleneglycol is from 2,000 to 10,000.

24. The process according to claim 23, characterised in that the average molecular weight of the polyethyleneglycol is in the range from 4,000 to 8,000.

25. The process according to claim 1, characterised in that the elution buffer is water.

26. (canceled)
